(19) United States
(12) Patent Application Publication
(10) Pub. No.: US 2013/0217015 A1
(43) Pub. Date: Aug. 22, 2013

(54) HMGA2 AS A BIOMARKER FOR DIAGNOSIS AND PROGNOSIS OF OVARIAN CANCER

(71) Applicants: Santo V. Nicosia, Tampa, FL (US); Patricia A. Kruk, Tampa, FL (US)

(72) Inventors: Santo V. Nicosia, Tampa, FL (US); Patricia A. Kruk, Tampa, FL (US)

(73) Assignee: UNIVERSITY OF SOUTH FLORIDA, Tampa, FL (US)

(21) Appl. No.: 13/808,813

(22) PCT Filed: Jul. 7, 2011
(86) PCT No.: PCT/US2011/043135

§ 371 Date: Apr. 25, 2013

Related U.S. Application Data
(60) Provisional application No. 61/362,598, filed on Jul. 8, 2010.

(51) Int. Cl. G01N 33/68

(52) U.S. Cl. CPC G01N 33/689
USPC 435/6.11; 436/501; 435/7.92; 435/6.12; 435/287.2; 422/69

(57) ABSTRACT

The subject invention pertains to uses of HMGA2 as a diagnostic biomarker for ovarian cancer and for prediction of a subject's resistance to cancer therapy. The methods of the subject invention are particular useful for early detection of epithelial ovarian cancer.

### CROSS-REFERENCE TO RELATED APPLICATION

This application claims the benefit of U.S. provisional application Ser. No. 61/362,598, filed Jul. 8, 2010, which is herein incorporated by reference in its entirety.

### BACKGROUND OF THE INVENTION

Ovarian cancer is the most common and lethal gynecologic neoplasm, affecting over 21,000 women annually. Due to the lack of reliable diagnostic assays, 70% of ovarian cancer patients are diagnosed at advanced stages and die within five years.

Currently, common diagnostic methods for detection of ovarian cancer include bimanual rectovaginal pelvic examination, determination of serum levels of CA 125, and transvaginal ultrasonography (TVS). Pelvic examination, a routine gynecologic examination, lacks sensitivity and specificity. Serum levels of CA 125, an ovarian-related antigenic determinant, can be detected by immunoassay. Although serum levels of CA 125 is elevated in 80% of epithelial ovarian cancer patients, its use for early detection of ovarian cancer is limited because only half of the patients with stage I ovarian cancer exibit elevated CA 125 levels. In addition, CA 125 lacks specificity as a diagnostic biomarker because its level may be elevated in a significant number of healthy women as well as women with benign ovarian lesions. TVS is a sensitive method for detecting ovarian lesions, however, its use as a single screening modality is limited due to the lack of specificity. Although the pairing of CA 125 and TVS for detection of ovarian cancer has improved specificity, this procedure produces false positive results in diagnosis of epithelial ovarian cancer (EOC). Thus, improved methods for early detection of ovarian cancer, especially epithelial ovarian cancer, are needed.

### BRIEF SUMMARY OF THE INVENTION

The aforementioned need is satisfied by the subject invention, providing a simple, safe, sensitive, specific and reliable method for early detection of ovarian cancer. In an embodiment, the subject invention provides uses of HMGA2 as a diagnostic biomarker for ovarian cancer. In another embodiment, the subject invention provides uses of HMGA2 as a biomarker for predicting patient resistance to cancer therapy. The methods of the subject invention are particular useful for early detection of epithelial ovarian cancer.

In one aspect, the subject invention provides a method of diagnosing whether a subject has ovarian cancer. In an embodiment, the method comprises:

a) obtaining a biological sample from a subject;

b) determining a level of high-mobility group AT-hook 2 (HMGA2) in the sample;

c) comparing the subject's HMGA2 to a predetermined reference value,

wherein the predetermined reference value is determined based on HMGA2 levels in a population, and

wherein an elevated HMGA2 level in the sample as compared to the predetermined reference value indicates that the subject has ovarian cancer.

In another aspect, the subject invention provides a method of evaluating the likelihood that an ovarian cancer subject will benefit from anti-cancer therapy. In one embodiment, the subject invention provides a method of determining whether the subject is resistant to cancer therapy, comprising:

a) obtaining a biological sample from a subject;

b) determining a level of high-mobility group AT-hook 2 (HMGA2) in the sample;

c) comparing the subject's HMGA2 to a predetermined reference value,

wherein the predetermined reference value is determined based on HMGA2 levels in a population, and

wherein an elevated HMGA2 level in the sample as compared to the predetermined reference value indicates that the subject is resistant to cancer therapy.

In another embodiment, the subject invention provides a method of evaluating the likelihood that an ovarian cancer subject will benefit from anti-cancer therapy, comprising: determining a level of high-mobility group AT-hook 2 (HMGA2) in a biological sample obtained from a subject that has ovarian cancer; wherein the determination is made at multiple times to monitor the change over time, wherein a progressive increase in the level of HMGA2 over time indicates a lessening likelihood that the subject will benefit from anti-cancer therapy.

In another aspect, the subject invention provides kits for diagnosis and prognosis of ovarian cancer. In one embodiment, the subject invention provides a kit for diagnosing whether a subject has ovarian cancer, wherein the kit comprises an agent that detects HMGA2 expression. In another embodiment, the subject invention provides a kit for evaluating the likelihood that an ovarian cancer subject will benefit from anti-cancer therapy, wherein the kit comprises an agent that detects HMGA2 expression. In one embodiment, the kits of the subject invention do not comprise an agent that detects HMGB1 expression.

In another aspect, the present invention relates to a device for the rapid detection of HMGA2 in a bodily fluid such as blood or urine. Preferably, the device is a lateral flow device. In one embodiment, the device comprises an application zone for receiving a sample of bodily fluid such as blood or urine; a labeling zone containing a binding agent that binds to HMGA2 in the sample; and a detection zone where HMGA2-bound binding agent is retained to give a signal, wherein the signal given for a sample from a subject with a HMGA2 level lower than a threshold concentration is different from the signal given for a sample from a patient with a HMGA2 level equal to or greater than a threshold concentration.

In another aspect, the invention relates to a simple, rapid, reliable, accurate and cost effective test for HMGA2 in a bodily fluid such as blood or urine, similar to currently available in-home pregnancy tests that could be used by subjects at home, in a physicians' office, or at a patient's bedside.

In one embodiment, the test is a method for measuring HMGA2 in a bodily fluid, comprising: (a) obtaining a sample of bodily fluid, such as blood or urine, from a subject; (b) contacting the sample with a binding agent that binds to any HMGA2 in the sample; (c) separating HMGA2-bound binding agent; (d) detecting a signal associated with the separated binding agent from (c); and (e) comparing the signal detected in step (d) with a reference signal which corresponds to the signal given by a sample from a subject with a HMGA2 level equal to a threshold concentration.

### BRIEF DESCRIPTION OF THE DRAWINGS

FIG. 1 shows endogenous HMGA2 expression in cell lysates of immortalized ovarian surface epithelial cells (HOSE, MCC3) and epithelial ovarian cancer (EOC) cell lines (A2780s, A2780cp, OV2008, C13, OVCAR5, SW626, TOV12D) maintained in culture.

FIG. 2 shows urinary levels of HGMA2 in healthy women and in two groups of patients with EOC. Urinary HMGA2 levels in pooled samples of cancer patients were approximately 1.5 fold higher than those of positive controls (C13 cell line lysate) and approximately 10-fold higher than those of healthy volunteers.

FIG. 3 shows urinary levels of HMGB1 in healthy controls and ovarian cancer patients.

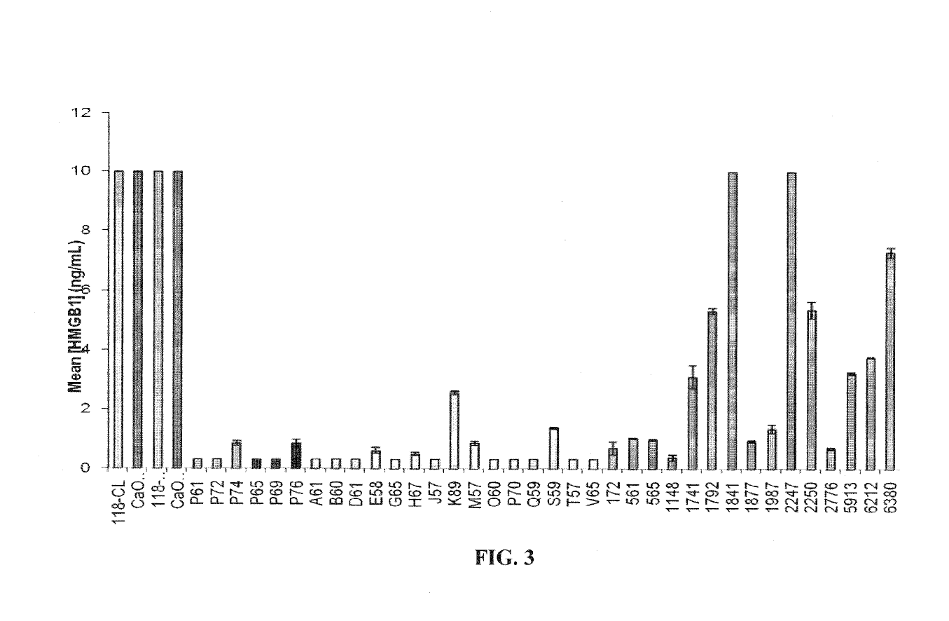

FIG. 4 shows results of receiver operating characteristic (ROC) analyses, indicating 91.56% area under this curve (AUC) with only 55% sensitivity at 88% specificity, which is at a level below sufficient stringency for use of HMGB1 as a diagnostic biomarker.

### BRIEF DESCRIPTION OF THE SEQUENCES

SEQ ID NO:1 is an amino acid sequence of human high-mobility group AT-hook 2 (HMGA2) polypeptide useful according to the subject invention.

SEQ ID NO:2 is a nucleotide sequence of human high-mobility group AT-hook 2 (HMGA2) nucleic acid useful according to the subject invention.

SEQ ID NO:3 is an amino acid sequence of human high-mobility group protein B1 (HMGB1) polypeptide useful according to the subject invention.

SEQ ID NO:4 is a nucleotide sequence of human high-mobility group protein B1 (HMGB1) nucleic acid useful according to the subject invention.

### DETAILED DISCLOSURE OF THE INVENTION

The subject invention provides uses of high-mobility group AT-hook 2 (HMGA2) as a diagnostic biomarker for ovarian cancer. The subject invention also provides uses of HMGA2 as a biomarker for predicting patient resistance to cancer therapy. Advantageously, the subject diagnostic and prognostic method is simple, safe, sensitive, specific, and reliable.

The subject invention is based, at least in part, on the surprising discovery that HMGA2 expression levels are markedly elevated in epithelial ovarian cancer cells and in ovarian cancer patients. Although urinary levels of high-mobility group protein B1 (HMGB1), another member of the HMG protein family, also appear elevated in ovarian cancer patients, HMGB1 appears to be a less sensitive biomarker than HMGA2 for detection of ovarian cancer.

In addition, Western blot results reveal that drug-resistant ovarian cancer cell lines exhibited increased cellular levels of HMGA2, as compared to the drug-sensitive EOC cell lines from which they were derived. Progressively increasing cellular levels of HMGA2 parallel the development of drug-resistance in ovarian cancer patients. Thus, an elevated HMGA2 level indicates that the patient is resistant to cancer therapy. Additionally, a progressive increase in HMGA2 levels indicates an increase in patient resistance to cancer therapy.

The methods of the subject invention are useful for diagnosis and prognosis of ovarian cancer, in particular, epithelial ovarian cancer. In an embodiment, the subject methods are useful for detecting early-stage ovarian cancer in a subject. In another embodiment, the subject methods are useful for early detection of ovarian cancer, in particular, stage I epithelial ovarian cancer.

In one aspect, the subject invention provides a method of diagnosing whether a subject has ovarian cancer. In an embodiment, the method comprises:

a) obtaining a biological sample from a subject;

b) determining a level of high-mobility group AT-hook 2 (HMGA2) in the sample;

c) comparing the subject's HMGA2 to a predetermined reference value,

wherein the predetermined reference value is determined based on HMGA2 levels in a population, and

wherein an elevated HMGA2 level in the sample compared to the predetermined reference value indicates that the subject has ovarian cancer. In one embodiment, the predetermined reference value is determined based on HMGA2 levels in a control population that do not have ovarian cancer. In an embodiment, the predetermined reference value is representative of the HMGA2 levels in a control population that do not have ovarian cancer.

In another aspect, the subject invention provides a method of evaluating the likelihood that an ovarian cancer subject will benefit from anti-cancer therapy. In one embodiment, the subject invention provides a method of determining whether the subject is resistant to cancer therapy, comprising:

a) obtaining a biological sample from a subject;

b) determining a level of high-mobility group AT-hook 2 (HMGA2) in the sample;

c) comparing the subject's HMGA2 to a predetermined reference value,

wherein the predetermined reference value is determined based on HMGA2 levels in a population, and

wherein an elevated HMGA2 level in the sample compared to the predetermined reference value indicates that the subject is resistant to cancer therapy. In one embodiment, an elevated HMGA2 level in the sample compared to the predetermined reference value indicates a low likelihood that the subject will benefit from anti-cancer therapy. In one embodiment, the predetermined reference value is determined based on HMGA2 levels in ovarian cancer subjects that are sensitive to anti-cancer therapy. Further, the determination can be optionally made at multiple times to monitor the change over time. An increase in levels of HMGA2 indicates worsening of ovarian cancer in a subject and/or an increase in resistance to cancer therapy.

In another embodiment, the subject invention provides a method of evaluating the likelihood that an ovarian cancer subject will benefit from anti-cancer therapy, comprising: determining a level of HMGA2 in a biological sample obtained from a subject that has ovarian cancer; wherein the determination is made at multiple times to monitor the change over time, wherein a progressive increase in the level of HMGA2 over time indicates a lessening likelihood that the subject will benefit from anti-cancer therapy.

In one embodiment, anti-cancer therapies to which an ovarian patient's response (e.g., sensitive or resistant) can be predicted in accordance with the current invention include, but are not limited to, chemotherapy and radiotherapy. Chemotherapeutic agents to which an ovarian patient's response (e.g., sensitive or resistant) can be predicted in accordance with the current invention include, but are not limited to, cisplatin, actinomycin, idarubicin, valrubicin, vincristine, doxorubicin, daunorubicin, mitoxantrone, vinblastine, vindesin, epirubicin, harringtonine, etoposide, bleomycin, mitomycin, teniposide, plicamycin, L-asparaginase, and cyclophosphamide.

The term “subject,” as used herein, describes an organism, including mammals such as primates. Mammalian species that can benefit from the subject methods include, but are not limited to, apes, chimpanzees, orangutans, humans, monkeys; and domesticated and/or laboratory animals such as dogs, cats, horses, cattle, pigs, sheep, goats, chickens, mice, rats, guinea pigs, and hamsters. Typically, the subject is a human.

The term “biological sample,” as used herein, includes but is not limited to a sample containing tissues, cells, and/or biological fluids isolated from a subject. Examples of biological samples include hut, are not limited to, tissues, cells, biopsies, blood, lymph, serum, plasma, urine, saliva, and tears. In a specific embodiment, the biological sample is a urinary sample. In another specific embodiment, the biological sample is a tissue (e.g., ovarian tissue) sample. In another specific embodiment, the biological sample is a blood (including whole blood, plasma) sample.

The “predetermined reference value” or “threshold concentration” (these terms are used interchangeably herein) can be established by skilled healthcare practitioners. For instance, the predetermined reference value can be established by measuring the levels of the biomarker in a normal population sample and correlating such levels with factors such as the incidence, severity, and/or frequency of developing ovarian cancer, more specifically, epithelial ovarian cancer. Thus, a subject's HMGA2 level as compared against the HMGA2 level in a normal population can be indicative of whether the subject has ovarian cancer. Further, the predetermined reference value is preferably established by using the same assay technique as is used for measurement of the subject's biomarker level, to avoid any error in standardization.

In one embodiment, the subject invention does not comprise the step of determining the HMGB1 expression level in the subject's biological sample.

In certain embodiments, the HMGA2 and HMGB1 expression levels are HMGA2 and HMGB1 transcript and/or protein levels. In one embodiment, a level of HMGA2 transcript (such as mRNA) is determined. In one embodiment, a level of HMGA2 protein level is determined.

In one embodiment, the HMGA2 level is a transcript level of HMGA2 sequence of SEQ ID NO:2. In certain embodiments, the HMGA2 level is a transcript level of HMGA2 sequence having at least 70%, 80%, 85%, 90%, 93%, 95%, 97%, 98%, or 99% identity to SEQ ID NO:2. In one embodiment, the HMGA2 level is a protein level of HMGA2 sequence that is SEQ ID NO:1 . In certain embodiments, the HMGA2 level is a protein level of HMGA2 sequence having at least 70%, 80%, 85%, 90%, 93%, 95%, 97%, 98%, or 99% identity to SEQ ID NO:1.

In one embodiment, the HMGB1 level is a transcript level of HMGB1 sequence of SEQ ID NO:4. In certain embodiments, the HMGB1 level is a transcript level of HMGA2 sequence having at least 70%, 80%, 85%, 90%, 93%, 95%, 97%, 98%, or 99% identity to SEQ ID NO:4. In one embodiment, the HMGB1 level is a protein level of HMGB1 sequence that is SEQ ID NO:3. In certain embodiments, the HMGB1 level is a protein level of HMGB1 sequence having at least 70%, 80%, 85%, 90%, 93%, 95%, 97%, 98%, or 99% identity to SEQ ID NO:3.

Methods for detecting biomarkers of the subject invention are well known in the art, including but not limited to, Western blots, Northern blots, Southern blots, enzyme-linked immunosorbent assays (ELISA), polymerase chain reaction (PCR), immunoprecipitation, immunofluorescence, radioimmunoassays, flow cytometry, immunocytochemistry, nucleic acid hybridization techniques, nucleic acid reverse transcription methods, and nucleic acid amplification methods.

In another aspect, the subject invention provides kits for diagnosis and prognosis of ovarian cancer. In one embodiment, the subject invention provides a kit for diagnosing whether a subject has ovarian cancer, wherein the kit comprises an agent that detects HMGA2 expression. In another embodiment, the subject invention provides a kit for evaluating the likelihood that an ovarian cancer subject will benefit from anti-cancer therapy, wherein the kit comprises an agent that detects HMGA2 expression. In certain embodiments, the kits of the subject invention do not comprise an agent that detects HMGB1 expression.

In one embodiment, agents that are capable of detecting HMGA2 in the biological samples of subjects are those that interact or bind with the HMGA2 polypeptide or the HMGA2 transcript mRNA. Examples of such agents (also referred to herein as binding agents) include, but are not limited to, HMGA2 antibodies or fragments thereof that bind HMGA2, HMGA2 binding partners, HMGA2-specific aptamers, and nucleic acid molecules that hybridize to the HMGA2 transcript mRNA. Preferably, the binding agent is labeled with a detectable substance (e.g., a detectable moiety). The binding agent may itself function as a label.

Antibodies applicable according to the present invention can be in various forms, including a whole immunoglobulin, an antibody fragment such as Fab, Fab′, F(ab′)₂, Fv region containing fragments, and similar fragments, as well as a single chain antibody that includes the variable domain complementarity determining regions (CDR), and similar forms. Antibodies within the scope of the invention can be of any isotype, including IgG, IgA, IgE, IgD, and IgM. IgG isotype antibodies can be further subdivided into IgG1, IgG2, IgG3, and IgG4 subtypes. IgA antibodies can be further subdivided into IgA1 and IgA2 subtypes.

As used herein, “stringent” conditions for hybridization refers to conditions whereby hybridization is typically carried out overnight at 20-25° C. below the melting temperature (Tm) of the DNA hybrid in 6×SSPE, 5× Denhardt's solution, 0.1% SDS, 0.1 mg/ml denatured DNA. The melting temperature, Tm, is described by the following formula (Beltz et al., 1983):

Tm=81.5C+16.6 Log[Na+]+0.41(% G+C)−0.61(% formamide)−600/length of duplex in base pairs.

Washes are typically carried out as follows:

(1) Twice at room temperature for 15 minutes in 1×SSPE, 0.1% SDS (low stringency wash).

(2) Once at Tm-20 C for 15 minutes in 0.2×SSPE, 0.1% SDS (moderate stringency wash).

“Specific binding” or “specificity” refers to the ability of an antibody or other agent to exclusively bind to an epitope presented on an antigen while having relatively little non-specific affinity with other proteins or peptides. Specificity can be relatively determined by binding or competitive binding assays, using, e.g., Biacore instruments. Specificity can be mathematically calculated by, e.g., an about 10:1, about 20:1, about 50:1, about 100:1, 10.000:1 or greater ratio of affinity/avidity in binding to the specific antigen versus nonspecific binding to other irrelevant molecules,

Optionally, the kit may include any material useful for performing any step of the subject invention as described above. For instance, the kit may further comprise any material useful for determination and/or analysis of HMGA2 expression levels. The kit may also comprise, e.g., a buffering agent, a preservative, or a stabilizing agent. Each component of the kit is usually enclosed within an individual container and all of the various containers are within a single package along with instructions. In one embodiment, the kit may comprise an article of manufacture for performing any step of the subject invention wherein the article of manufacture is a urine test strip. The terms “detecting” or “detect” include assaying or otherwise establishing the presence or absence of the target HMGA2 (HMGA2-encoding nucleic acid sequence or HMGA2 gene product (polypeptide)), subunits thereof, or combinations of agent bound targets, and the like, or assaying for, interrogating, ascertaining, establishing, or otherwise determining one or more factual characteristics of gynecological cancer, metastasis, stage, or similar conditions. The term encompasses diagnostic, prognostic, and monitoring applications for HMGA2 and other cancer biomarkers. The term encompasses quantitative, semi-quantitative, and qualitative detection methodologies. In embodiments of the invention involving detection of HMGA2 protein (as opposed to nucleic acid molecules encoding HMGA2 protein), the detection method is preferably an ELISA-based method. Preferably, in the various embodiments of the invention, the detection method provides an output (i.e., readout or signal) with information concerning the presence, absence, or amount of HMGA2 in a sample from a subject. For example, the output may be qualitative (e.g., “positive” or “negative”), or quantitative (e.g., a concentration such as nanograms per milliliter).

In an embodiment, the invention relates to a method for detecting cancer in a subject by qualitatively or quantitatively detecting HMGA2 protein or encoding nucleic acids (DNA or RNA) in a biological sample such as urine from the subject, comprising (a) contacting (reacting) the biological sample with a binding agent specific for HMGA2 which is directly or indirectly labeled with a detectable substance; and (b) detecting the detectable substance.

In an embodiment, the invention relates to a method for diagnosing and/or monitoring cancer in a subject by quantitating HMGA2 in a biological sample, such as urine or blood, from the subject, comprising (a) reacting the biological sample with a binding agent specific for HMGA2 which is directly or indirectly labeled with a delectable substance; and (b) detecting the detectable substance.

Embodiments of the methods of the invention involve (a) contacting a biological sample from a subject with a binding agent specific for HMGA2 which is directly or indirectly labeled with an enzyme; (b) adding a substrate for the enzyme wherein the substrate is selected so that the substrate, or a reaction product of the enzyme and substrate, forms fluorescent complexes; (c) quantitating HMGA2 in the sample by measuring fluorescence of the fluorescent complexes; and (d) comparing the quantitated levels to that of a standard.

A preferred embodiment of the invention comprises the following steps:

(a) incubating a biological sample with a first antibody specific for HMGA2 which is directly or indirectly labeled with a detectable substance, and a second antibody specific for HMGA2 which is immobilized;

(b) separating the first antibody from the second antibody to provide a first antibody phase and a second antibody phase;

(c) detecting the detectable substance in the first or second antibody phase thereby quantitating HMGA2 in the biological sample; and

(d) comparing the quantitated HMGA2 with a standard.

A standard used in a method of the invention may correspond to HMGA2 levels obtained for samples from healthy control subjects, from subjects with benign disease (e.g., benign gynecological disease), subjects with early stage gynecological cancer, or from other samples of the subject. Increased levels of HMGA2 as compared to the standard may be indicative of cancer, such as early or late stage ovarian cancer.

The methods of the invention are particularly useful in the diagnosis of early stage ovarian cancer (e.g., when the subject is asymptomatic) and for the prognosis of ovarian cancer disease progression and mortality. As illustrated herein, increased levels of HMGA2 detected in a sample (e.g., urine, serum, plasma, whole blood, ascites) compared to a standard (e.g., threshold concentration levels for normal or benign disorders) are indicative of advanced disease stage, serous histological type, suboptimal debulking, large residual tumor, and/or increased risk of disease progression and mortality.

In embodiments of the invention, the method described herein is adapted for diagnosing and monitoring gynecological cancer by quantitating HMGA2 in biological samples from a subject. Preferably, the amount of HMGA2 quantitated in a sample from a subject being tested is compared to levels quantitated for another sample or an earlier sample from the subject, or levels quantitated for a control sample. Levels for control samples from healthy subjects may be established by prospective and/or retrospective statistical studies. Healthy subjects who have no clinically evident disease or abnormalities may be selected for statistical studies. Diagnosis may be made by a finding of statistically different levels of HMGA2 compared to a control sample or previous levels quantitated for the same subject.

In certain embodiments, the methods of the invention can be carried out on a solid support. The solid supports used may be those which are conventional for the purpose of assaying an analyte in a biological sample, and are typically constructed of materials such as cellulose, polysaccharide such as Sephadex, and the like, and may be partially surrounded by a housing for protection and/or handling of the solid support. The solid support can be rigid, semi-rigid, flexible, elastic (having shape-memory), etc., depending upon the desired application. HMGA2 can be detected in a sample in vivo or in vitro (ex vivo). When, according to an embodiment of the invention, the amount of HMGA2 in a sample is to be determined without removing the sample from the body (i.e., in vivo), the support should be one which is harmless to the subject and may be in any form convenient for insertion into an appropriate part of the body. For example, the support may be a probe made of polytetrafluoroethylene, polystyrene or other rigid non-harmful plastic material and having a size and shape to enable it to be introduced into a subject. The selection of an appropriate inert support is within the competence of those skilled in the art, as are its dimensions for the intended purpose.

A contacting step in the assay (method) of the invention can involve contacting, combining, or mixing the biological sample and the solid support, such as a reaction vessel, microvessel, tube, microtube, well, multi-well plate, or other solid support. In an embodiment of the invention, the solid support to be contacted with the biological sample (e.g., urine) has an absorbent pad or membrane for lateral flow of the liquid medium to be assayed, such as those available from Millipore Corp. (Bedford, Mass.), including but not limited to Hi-Flow Plus™ membranes and membrane cards, and SureWick™ pad materials.

The diagnostic device useful in carrying out the methods of the invention can be constructed in any form adapted for the intended use. Thus, in one embodiment, the device of the invention can be constructed as a disposable or reusable test strip or stick to be contacted with a biological sample such as urine or blood for which HMGA2 level is to be determined. In another embodiment, the device can be constructed using art recognized micro-scale manufacturing techniques to produce needle-like embodiments capable of being implanted or injected into an anatomical site, such as the peritoneal cavity, for indwelling diagnostic applications. In other embodiments, devices intended for repeated laboratory use can be constructed in the form of an elongated probe.

In preferred embodiments, the devices of the invention comprise a solid support (such as a strip or dipstick), with a surface that functions as a lateral flow matrix defining a flow path for a biological sample such as urine, whole blood, serum, plasma, peritoneal fluid, or ascites.

Immunochromatographic assays, also known as lateral flow test strips or simply strip tests, for detecting various analytes of interest, have been known for some time, and may be used for detection of HMGA2. The benefits of lateral flow tests include a user-friendly format, rapid results, long-term stability over a wide range of climates, and relatively low cost to manufacture. These features make lateral flow tests ideal for applications involving home testing, rapid point of care testing, and testing in the field for various analytes. The principle behind the test is straightforward. Essentially, any ligand that can be bound to a visually detectable solid support, such as dyed microspheres, can be tested for, qualitatively, and in many cases even semi-quantitatively. For example, a one-step lateral flow immunostrip for the detection of free and total prostate specific antigen in serum is described in Fernandez-Sanchez et al. (J. Immuno. Methods, 2005, 307(1-2):1-12, which is incorporated herein by reference) and may be adapted for detection of HMGA2 in a biological sample such as blood or urine.

Some of the more common immunochromatographic assays currently on the market are tests for pregnancy (as an over-the-counter (OTC) test kit), Strep throat, and Chlamydia. Many new tests for well-known antigens have been recently developed using the immunochromatographic assay method. For instance, the antigen for the most common cause of community acquired pneumonia has been known since 1917, but a simple assay was developed only recently, and this was done using this simple test strip method (Murdoch, D. R. et al. J Clin Microbial, 2001, 39:3495-3498). Human immunodeficiency virus (HIV) has been detected rapidly in pooled blood using a similar assay (Soroka, S. D. et al. J Clin Virol, 2003, 27:90-96). A nitrocellulose membrane card has also been used to diagnose schistosomiasis by detecting the movement and binding of nanoparticles of carbon (van Dam, G. J. et al. J Clin Microbial, 2004, 42:5458-5461).

The two common approaches to the immunochromatographic assay are the non-competitive (or direct) and competitive (or competitive inhibition) reaction schemes (TechNote #303, Rev. #001, 1999, Bangs Laboratories, Inc., Fishers, Ind.). The direct (double antibody sandwich) format is typically used when testing for larger analytes with multiple antigenic sites such as luteinizing hormone (LH), human chorionic gonadotropin (hCG), and HIV. In this instance, less than an excess of sample analyte is desired, so that some of the microspheres will not be captured at the capture line, and will continue to flow toward the second line of immobilized antibodies, the control zone. This control line uses species-specific anti-immunoglobulin antibodies, specific for the conjugate antibodies on the microspheres. Free antigen, if present, is introduced onto the device by adding sample (urine, serum, etc.) onto a sample addition pad. Free antigen then binds to antibody-microsphere complexes. Antibody 1, specific for epitope 1 of sample antigen, is coupled to dye microspheres and dried onto the device. When sample is added, microsphere-antibody complex is rehydrated and carried to a capture zone and control lines by liquid. Antibody 2, specific for a second antigenic site (epitope 2) of sample antigen, is dried onto a membrane at the capture line. Antibody 3, a species-specific, anti-immunoglobulin antibody that will react with antibody 1, is dried onto the membrane at the control line. If antigen is present in the sample (i.e., a positive test), it will bind by its two antigenic sites, to both antibody 1 (conjugated to microspheres) and antibody 2 (dried onto membrane at the capture line). Antibody 1-coated microspheres are bound by antibody 3 at the control line, whether antigen is present or not. If antigen is not present in the sample (a negative test), microspheres pass the capture line without being trapped, but are caught by the control line.

The competitive reaction scheme is typically used when testing for small molecules with single antigenic determinants, which cannot bond to two antibodies simultaneously. As with double antibody sandwich assay, free antigen, if present is introduced onto the device by adding sample onto a sample pad. Free antigen present in the sample binds to an antibody-microsphere complex. Antibody 1 is specific for sample antigen and couple to dyed microspheres. An antigen-carrier molecule (typically BSA) conjugate is dried onto a membrane at the capture line. Antibody 2 (Ab2) is dried onto the membrane at the control line, and is a species-specific anti-immunoglobulin that will capture the reagent particles and confirm that the test is complete. If antigen is present in the sample (a positive test), antibody on microspheres (Ab1) is already saturated with antigen from sample and, therefore, antigen conjugate bound at the capture line does not bind to it. Any microspheres not caught by the antigen carrier molecule can be caught by Ab2 on the control line. If antigen is not present in the sample (a negative test), antibody-coated dyed microspheres are allowed to be captured by antigen conjugate bound at the capture line.

Normally, the membranes used to hold the antibodies in place on these devices are made of primary hydrophobic materials, such as nitrocellulose. Both the microspheres used as the solid phase supports and the conjugate antibodies are hydrophobic, and their interaction with the membrane allows them to be effectively dried onto the membrane.

Samples and/or HMGA2-specific binding agents may be arrayed on the solid support, or multiple supports can be utilized, for multiplex detection or analysis. “Arraying” refers to the act of organizing or arranging members of a library (e.g., an array of different samples or an array of devices that target the same target molecules or different target molecules), or other collection, into a logical or physical array. Thus, an “array” refers to a physical or logical arrangement of, e.g., biological samples. A physical array can be any “spatial format” or physically gridded format” in which physical manifestations of corresponding library members are arranged in an ordered manner, lending itself to combinatorial screening. For example, samples corresponding to individual or pooled members of a sample library can be arranged in a series of numbered rows and columns, e.g., on a multi-well plate. Similarly, binding agents can be plated or otherwise deposited in microtitered, e.g., 96-well, 384-well, or-1536 well, plates (or trays). Optionally, HMGA2-specific binding agents may be immobilized on the solid support.

Detection of HMGA2 and cancer biomarkers, and other assays that are to be carried out on samples, can be carried out simultaneously or sequentially with the detection of other target molecules, and may be carried out in an automated fashion, in a high-throughput format.

The HMGA2-specific binding agents can be deposited but “free” (non-immobilized) in the conjugate zone, and be immobilized in the capture zone of a solid support. The HMGA2-specific binding agents may be immobilized by non-specific adsorption onto the support or by covalent bonding to the support, for example. Techniques for immobilizing binding agents on supports are known in the art and are described for example in U.S. Pat. Nos. 4,399,217, 4,381,291, 4,357,311, 4,343,312 and 4,260,678, which are incorporated herein by reference. Such techniques can be used to immobilize the binding agents in the invention. When the solid support is polytetrafluoroethylene, it is possible to couple hormone antibodies onto the support by activating the support using sodium and ammonia to aminate it and covalently bonding the antibody to the activated support by means of a carbodiimide reaction (yon Klitzing, Schultek, Strasburger, Fricke and Wood in “Radioimmunoassay and Related Procedures in Medicine 1982”, International Atomic Energy Agency, Vienna (1982), pages 57-62.).

The diagnostic device of the invention can utilize lateral flow strip (LFS) technology, which has been applied to a number of other rapid strip assay systems, such as over-the-counter early pregnancy test strips based on antibodies to human chorionic gonadotropin (hCG). As with many other diagnostic devices, the device utilizes a binding agent to bind the target molecule (HMGA2). The device has an application zone for receiving a biological sample such as blood or urine, a labeling zone containing label which binds to HMGA2 in the sample, and a detection zone where HMGA2 label is retained.

Binding agent retained in the detection zone gives a signal, and the signal differs depending on whether HMGA2 levels in the biological sample are lower than, equal to, or greater than a given threshold concentration. For example, in the case of urinary HMGA2 for the detection of ovarian cancer, the threshold concentration may be between 0 ng/ml and 2.0 ng/ml. In another embodiment, in the case of urinary HMGA2 for the detection of ovarian cancer, the threshold concentration is 1.8 ng/ml. A sample from a subject having a HMGA2 level equal to or greater than the given reference HMGA2 concentration can be referred to as a “threshold level”, “threshold amount”, or “threshold sample”. The application zone in the device is suitable for receiving the biological sample to be assayed. It is typically formed from absorbent material such as blotting paper. The labeling zone contains binding agent that binds to any HMGA2 in the sample. In one embodiment, the binding agent is an antibody (e.g., monoclonal antibody, polyclonal antibody, antibody fragment). For ease of detection, the binding agent is preferably in association with a label that provides a signal that is visible to the naked eye, e.g., it is tagged with a fluorescent tag or a colored tag such as conjugated colloidal gold, which is visible as a pink color.

The detection zone retains HMGA2 to which the binding agent has bound. This will typically be achieved using an immobilized binding agent such as an immobilized antibody. Where the binding agent in the labeling zone and the detection zone are both antibodies, they will typically recognize different epitopes on the target molecule (HMGA2 protein). This allows the formation of a “sandwich” comprising antibody-HMGA2-antibody.

The detection zone is downstream of the application zone, with the labelling zone typically located between the two. A sample will thus migrate from the application zone into the labeling zone, where any in the sample binds to the label. HMGA2-binding agent complexes continue to migrate into the detection zone together with excess binding agent. When the HMGA2-binding agent complex encounters the capture reagent, the complex is retained whilst the sample and excess binding agent continue to migrate. As HMGA2 levels in the sample increase, the amount of binding agent (in the form of HMGA2-binding agent complex) retained in the detection zone increases proportionally.

In preferred embodiments, the device of the invention has the ability to distinguish between samples according to the threshold concentration. This can be achieved in various ways.

One type of device includes a reference zone that includes a signal of fixed intensity against which the amount of binding agent retained in the detection zone can be compared—when the signal in the detection zone equals the signal in the reference zone, the sample is a standard or a threshold sample; when the signal in the detection zone is less intense than the reference zone, the sample contains less HMGA2 than a threshold sample; when the signal in the detection zone is more intense than the reference zone, the sample contains more HMGA2 than a threshold sample.

A suitable reference zone can be prepared and calibrated without difficulty. For this type of device, the binding agent will generally be present in excess to HMGA2 in the sample, and the reference zone may be upstream or, preferably, downstream of the detection zone. The signal in the reference zone will be of the same type as the signal in the detection zone, i.e., they will typically both be visible to the naked eye, e.g., they will use the same tag. A preferred reference zone in a device of this type comprises immobilized protein (e.g., bovine serum albumin) which is tagged with colloidal gold.

In another device of the invention, the reference zone is downstream of the detection zone and includes a reagent which captures binding agent (e.g., an immobilised anti-binding agent antibody). Binding agent that flows through the device is not present in excess, but is at a concentration such that 50% of it is bound by a sample having HMGA2 at the threshold concentration. In a threshold sample, therefore, 50% of the binding agent will be retained in the detection zone and 50% in the reference zone. If the HMGA2 level in the sample is greater than in a threshold sample, less than 50% of the binding agent will reach the reference zone and the detection zone will give a more intense signal than the reference zone; conversely, if the HMGA2 level in the sample is less than in a threshold sample, less than 50% of the binding agent will be retained in the detection zone and the reference zone will give a more intense signal than the detection zone.

In another device of the invention which operates according to similar principles, the reference zone is downstream of the detection zone and includes a limiting amount of a reagent which captures binding agent (e.g., an immobilised anti-binding agent antibody). The reagent is present at a level such that it retains the same amount of label which would bind to the detection zone for a threshold sample, with excess label continuing to migrate beyond the reference zone.

In these three types of device, therefore, a comparison between the detection zone and the reference zone is used to compare the sample with the threshold concentration. The detection:reference binding ratio can preferably be determined by eye. Close juxtaposition of the detection and reference zones is preferred in order to facilitate visual comparison of the signal intensities in the two zones.

In a fourth type of device, no reference zone is needed, but the detection zone is configured such that it gives an essentially on/off response, e.g., no signal is given below the threshold concentration but, at or above the threshold, signal is given.

In a fifth type of device, no reference zone is needed, but an external reference is used which corresponds to the threshold concentration. This can take various forms, e.g., a printed card against which the signal in the detection zone can be compared, or a machine reader which compares an absolute value measured in the detection zone (e.g., a calorimetric signal) against a reference value stored in the machine.

In some embodiments of the invention, the device includes a control zone downstream of the detection zone. This will generally be used to capture excess binding agent that passes through the detection and/or reference zones (e.g., using immobilized anti-binding agent antibody). When binding agent is retained at the control zone, this confirms that mobilization of the binding agent and migration through the device have both occurred. It will be appreciated that this function may be achieved by the reference zone.

In a preferred embodiment, the detection, reference and control zones are preferably formed on a nitrocellulose support.

Migration from the application zone to the detection zone will generally be assisted by a wick downstream of the detection zone to aid capillary movement. This wick is typically formed from absorbent material such as blotting or chromatography paper.

The device of the invention can be produced simply and cheaply, conveniently in the form of a dipstick. Furthermore, it can be used very easily, for instance by the home user. The invention thus provides a device which can be used at home as a screen for cancer, such as ovarian cancer.

In one aspect, the present invention includes kits comprising the required elements for diagnosing or monitoring cancer. Preferably, the kits comprise a container for collecting biological fluid from a patient and an agent for detecting the presence of HMGA2 or its encoding nucleic acid in the fluid. The components of the kits can be packaged either in aqueous medium or in lyophilized form.

The methods of the invention can be carried out using a diagnostic kit for qualitatively or quantitatively detecting HMGA2 in a sample such as blood or urine. By way of example, the kit can contain binding agents (e.g., antibodies) specific for HMGA2, antibodies against the antibodies labeled with an enzyme; and a substrate for the enzyme. The kit can also contain a solid support such as microtiter multi-well plates, standards, assay diluent, wash buffer, adhesive plate covers, and/or instructions for carrying out a method of the invention using the kit. In one embodiment, the kit includes one or protease inhibitors (e.g., a protease inhibitor cocktail) to be applied to the biological sample to be assayed (such as blood or urine).

Kits for diagnosing or monitoring gynecological cancer containing one or more agents that detect the HMGA2 protein, such as but not limited to HMGA2 antibodies, fragments thereof, or HMGA2 binding partners, can be prepared. The agent(s) can be packaged with a container for collecting the biological fluid from a patient. When the antibodies or binding partner are used in the kits in the form of conjugates in which a label is attached, such as a radioactive metal ion or a moiety, the components of such conjugates can be supplied either in fully conjugated form, in the form of intermediates or as separate moieties to be conjugated by the user of the kit.

Kits containing one or more agents that detect HMGA2 nucleic acid, such as but not limited to the full length HMGA2 nucleic acid, HMGA2 oligonucleotides, and pairs of HMGA2 primers can also be prepared. The agent(s) can be packaged with a container for collecting biological samples from a patient. The nucleic acid can be in the labeled form or to be labeled form.

Other components of the kit may include but are not limited to, means for collecting biological samples, means for labeling the detecting agent (binding agent), membranes for immobilizing the HMGA2 protein or HMGA2 nucleic acid in the biological sample, means for applying the biological sample to a membrane, means for binding the agent to HMGA2 in the biological sample of a subject, a second antibody, a means for isolating total RNA from a biological fluid of a subject, means for performing gel electrophoresis, means for generating cDNA from isolated total RNA, means for performing hybridization assays, and means for performing PCR, etc.

As used herein, the term “ELISA” includes an enzyme-linked immunoabsorbent assay that employs an antibody or antigen bound to a solid phase and an enzyme-antigen or enzyme-antibody conjugate to detect and quantify the amount of an antigen (e.g., HMGA2) or antibody present in a sample. A description of the ELISA technique is found in Chapter 22of the 4^{th} Edition of Basic and Clinical Immunology by D. P. Sites et al., 1982, published by Lange Medical Publications of Los Altos, Calif. and in U.S. Pat. Nos. 3,654,090; 3,850,752; and 4,016,043, the disclosures of which are herein incorporated by reference. ELISA is an assay that can be used to quantitate the amount of antigen, proteins, or other molecules of interest in a sample. In particular, ELISA can be carried out by attaching on a solid support (e.g., polyvinylchloride) an antibody specific for an antigen or protein of interest. Cell extract or other sample of interest such as urine can be added for formation of an antibody-antigen complex, and the extra, unbound sample is washed away. An enzyme-linked antibody, specific for a different site on the antigen is added. The support is washed to remove the unbound enzyme-linked second antibody. The enzyme-linked antibody can include, but is not limited to, alkaline phosphatase. The enzyme on the second antibody can convert an added colorless substrate into a colored product or can convert a non-fluorescent substrate into a fluorescent product. The ELISA-based assay method provided herein can be conducted in a single chamber or on an array of chambers and can be adapted for automated processes.

In these exemplary embodiments, the antibodies can be labeled with pairs of FRET dyes, bioluminescence resonance energy transfer (BRET) protein, fluorescent dye-quencher dye combinations, beta gal complementation assays protein fragments. The antibodies may participate in FRET, BRET, fluorescence quenching or beta-gal complementation to generate fluorescence, colorimetric or enhanced chemiluminescence (ECL) signals, for example.

These methods are routinely employed in the detection of antigen-specific antibody responses, and are well described in general immunology text books such as Immunology by Ivan Roitt, Jonathan Brostoff and David Male (London: Mosby, c1998. 5th ed. and Immunobiology: Immune System in Health and Disease/Charles A. Janeway and Paul Travers. Oxford: Blackwell Sci. Pub., 1994), the contents of which are herein incorporated by reference.

As used herein, the terms solid “support”, “substrate”, and “surface” refer to a solid phase which is a porous or non-porous water insoluble material that can have any of a number of shapes, such as strip, rod, particle, beads, or multi-welled plate. In some embodiments, the support has a fixed organizational support matrix that preferably functions as an organization matrix, such as a microtiter tray. Solid support materials include, but are not limited to, cellulose, polysaccharide such as Sephadex, glass, polyacryloylmorpholide, silica, controlled pore glass (CPG), polystyrene, polystyrene/latex, polyethylene such as ultra high molecular weight polyethylene (UPE), polyamide, polyvinylidine fluoride (PVDF), polytetrafluoroethylene (PTFE; TEFLON), carboxyl modified teflon, nylon, nitrocellulose, and metals and alloys such as gold, platinum and palladium. The solid support can be biological, non-biological, organic, inorganic, or a combination of any of these, existing as particles, strands, precipitates, gels, sheets, pads, cards, strips, dipsticks, test strips, tubing, spheres, containers, capillaries, pads, slices, films, plates, slides, etc., depending upon the particular application. Preferably, the solid support is planar in shape, to facilitate contact with a biological sample such as urine, whole blood, plasma, serum, peritoneal fluid, or ascites fluid. Other suitable solid support materials will be readily apparent to those of skill in the art. The solid support can be a membrane, with or without a backing (e.g., polystyrene or polyester card backing), such as those available from Millipore Corp. (Bedford, Mass.), e.g., Hi-Flow™ Plus membrane cards. The surface of the solid support may contain reactive groups, such as carboxyl, amino, hydroxyl, thiol, or the like for the attachment of nucleic acids, proteins, etc. Surfaces on the solid support will sometimes, though not always, be composed of the same material as the support. Thus, the surface can be composed of any of a wide variety of materials, such as polymers, plastics, resins, polysaccharides, silica or silica-based materials, carbon, metals, inorganic glasses, membranes, or any of the aforementioned support materials (e.g., as a layer or coating).

As used herein, the terms “label” and “tag” refer to substances that may confer a detectable signal, and include, but are not limited to, enzymes such as alkaline phosphatase, glucose-6-phosphate dehydrogenase, and horseradish peroxidase, ribozyme, a substrate for a replicase such as QB replicase, promoters, dyes, fluorescers, such as fluorescein, isothiocynate, rhodamine compounds, phycoerythrin, phycocyanin, allophycocyanin, o-phthaldehyde, and fluorescamine, chemiluminescers such as isoluminol, sensitizers, coenzymes, enzyme substrates, radiolabels, particles such as latex or carbon particles, liposomes, cells, etc., which may be further labeled with a dye, catalyst or other detectable group.

Following are examples that illustrate procedures for practicing the invention. These examples should not be construed as limiting. All percentages are by weight and all solvent mixture proportions are by volume unless otherwise noted.

### EXAMPLE 1

### Elevated Levels of HMGA2 Expression in Epithelial Ovarian Cancer Cells

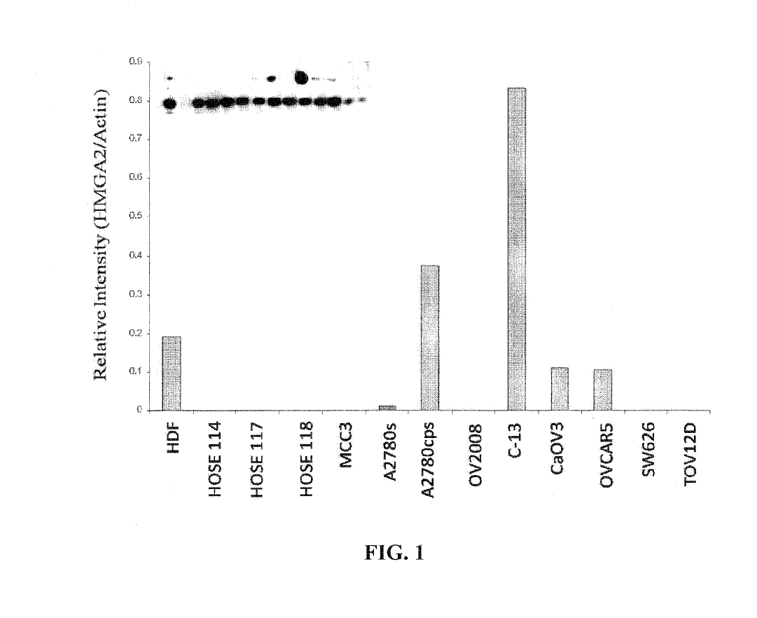

This Example shows that HMGA2 expression is upregulated in epithelial ovarian cancer cells, as compared to normal ovarian surface epithelial cells. Specifically, endogenous HMGA2 expression (Western immunoblot densitometry normalized to actin) in cell lysates of immortalized ovarian surface epithelial cells (HOSE, MCC3) and epithelial ovarian cancer (EOC) cell lines (A2780s, A2780cp, OV2008, C13, OVCAR5, SW626, TOV12D) maintained in culture was determined. The Western blot results are shown in inset (last 7 lanes, EOC cell lines) of FIG. 1. HMGA2 expression in human dermal fibroblasts (HDF) serves as a positive control. The results, as shown in FIG. 1, reveal that HMGA2 is highly expressed in 4/8 EOC lines, but is absent in normal ovarian surface epithelial cells. Specifically, HMGA2 levels were up to 100× higher in two EOC cell lines with acquired cisplatin-resistance (A2780cp and C13).

### EXAMPLE 2

### Elevated Levels of HMGA2 Expression in Epithelial Ovarian Cancer Patients

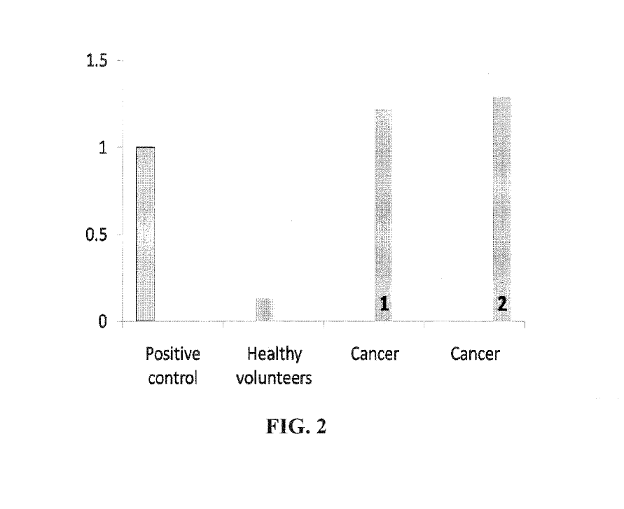

This Example shows that urinary levels of HGMA2 are elevated in patients with EOC, as compared to that of healthy women. Specifically, HGMA2 expression in C13 ovarian cancer cell lysate is used as a positive control with relative density value set at ‘1’ and HGMA2 expression in urinary samples are normalized to that of the C13 ovarian cancer cell lysate. The normal urine is a pool of 5 concentrated samples while cancer urines are each pools of 5 concentrated urinary samples of high grade serous EOC as limited amounts of urine necessitated pooling of individual samples for Western immunoblot analysis. A commercially available HMGA2 ELISA had previously been available, but was no longer available at the time of these assays. As shown in FIG. 2, HMGA2 protein levels in both cancer groups are higher than that of the + control and much higher than that of the healthy volunteers.

### EXAMPLE 3

### Lack of Sensitivity of HMGB1 for Use as a Diagnostic Biomarker for Ovarian Cancer

This Example reveals that HMGB1 lacks sensitivity for use as a diagnostic biomarker for ovarian cancer. Specifically, plasma and urinary levels of HMGB1, another member of the HMG protein family, were determined using a commercially available ELISA for HMGB1 (IBL International, Hamburg, Germany). Plasma and urinary HMGB1 levels in healthy controls and patients with ovarian cancer were compared. Cell lysates and conditioned medium from HOSE (118) and EOC cancer cell lines (CAOV3) were all equally positive for HMGB1 and served as positive controls for this assay.

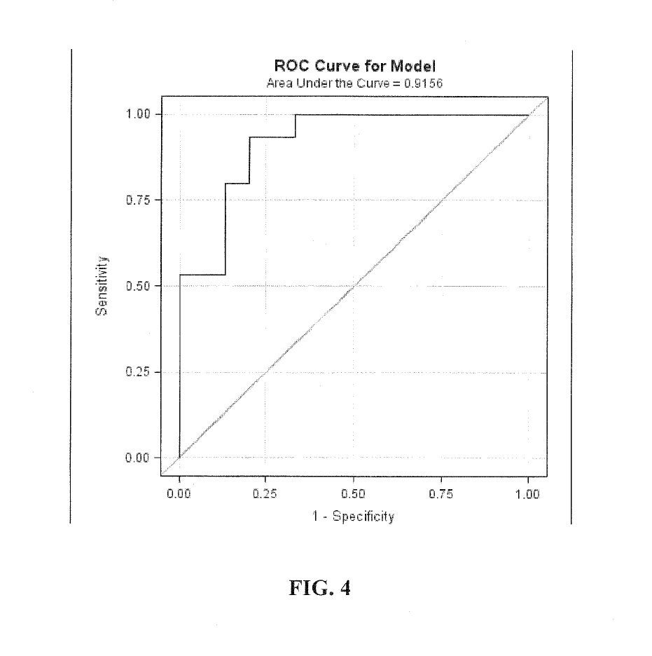

As shown in FIG. 2, no difference was observed among plasma levels of HMGB1 of healthy controls (light blue bars) and EOC patients (dark blue bars). The average levels of urinary HMGB1 of healthy controls (light teal bars) was 0.605 ng/ml and ranged from 0.313 ng/ml to 2.574 ng/ml. In contrast, the average urinary HMGB1 levels of EOC patients were 3.61 ng/ml and ranged from 0.381 ng/ml to >10 ng/ml (exceeded the standard curve values). This 6× increase in urinary HMGB1 in EOC as compared to healthy controls is statistically significant (p<0.005). However, receiver operating characteristic (ROC) analyses, as shown in FIG. 4, indicated 91.56% area under this curve (AUC) with only 55% sensitivity at 88% specificity, which is at a level below sufficient stringency for use as a diagnostic biomarker.

All patents, patent applications, provisional applications, and publications referred to or cited herein are incorporated by reference in their entirety, including all figures and tables, to the extent they are not inconsistent with the explicit teachings of this specification.

It should be understood that the examples and embodiments described herein are for illustrative purposes only and that various modifications or changes in light thereof will be suggested to persons skilled in the art and are to be included within the spirit and purview of this application. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

We claim:

1. A method of diagnosing whether a subject has ovarian cancer, comprising:
a) obtaining a biological sample from a subject;
b) determining a level of high-mobility group AT-hook 2 (HMGA2) in the sample; and
c) comparing the subject's HMGA2 to a predetermined reference value,
wherein the predetermined reference value is based on HMGA2 levels in a control population that do not have ovarian cancer, and
wherein an elevated HMGA2 level in the sample compared to the predetermined reference value indicates that the subject has ovarian cancer.

2. The method, according to claim 1, wherein the biological sample is a urine, blood, or tissue sample.

3. The method, according to claim 1, wherein the HMGA2 level is determined using Western immunoblot or enzyme-linked immunosorbent assay (ELISA).

4. The method, according to claim 1, wherein the ovarian cancer is epithelial ovarian cancer.

5. A method of evaluating the likelihood that an ovarian cancer subject will benefit from anti-cancer therapy, comprising:
a) obtaining a biological sample from a subject that has ovarian cancer;
b) determining a level of high-mobility group AT-hook 2 (HMGA2) in the sample; and
c) comparing the subject's HMGA2 to a predetermined reference value,
wherein the predetermined reference value is based on HMGA2 levels in ovarian cancer subjects that are sensitive to anti-cancer therapy, and
wherein an elevated HMGA2 level in the sample compared to the predetermined reference value indicates a low likelihood that the subject will benefit from anti-cancer therapy.

6. The method, according to claim 5, wherein the biological sample is a urine, blood, or tissue sample.

7. The method, according to claim 5, wherein the HMGA2 level is determined using Western immunoblot or enzyme-linked immunosorbent assay (ELISA).

8. The method, according to claim 5, wherein the ovarian cancer is epithelial ovarian cancer.

9. The method, according to claim 5, wherein the anti-cancer therapy is selected from chemotherapy, radiotherapy, or a combination thereof.

10. The method, according to claim 9, wherein the chemotherapy comprises administering to the subject a chemotherapeutic agent selected from the group consisting of cisplatin, actinomycin, idarubicin, valrubicin, vincristine, doxorubicin, daunorubicin, mitoxantrone, vinblastine, vindesin, epirubicin, harringtonine, etoposide, bleomycin, mitomycin, teniposide, plicamycin, L-asparaginase, and cyclophosphamide.

11. A method of evaluating the likelihood that an ovarian cancer subject will benefit from anti-cancer therapy, comprising: determining a level of high-mobility group AT-hook 2 (HMGA2) in a biological sample obtained from a subject that has ovarian cancer; wherein the determination is made at multiple times to monitor the change over time, wherein a progressive increase in the level of HMGA2 over time indicates a low likelihood that the subject will benefit from anti-cancer therapy.

12. The method, according to claim 11, wherein the biological sample is a urine, blood, or tissue sample.

13. The method, according to claim 11, wherein the HMGA2 level is determined using Western immunoblot or enzyme-linked immunosorbent assay (ELISA).

14. The method, according to claim 11, wherein the ovarian cancer is epithelial ovarian cancer.

15. A kit for diagnosing whether a subject has ovarian cancer, comprising an agent for detection of HMGA2 expression and printed instructions for detecting the HMGA2 level in a biological sample, wherein the kit comprises an HMGA2 binding agent and does not comprise an agent that binds HMGB1.

16. The kit, according to claim 15, wherein the agent for detection of HMGA2 expression is an anti-HMGA2 antibody and/or a nucleic acid that specifically binds to an HMGA2 transcript mRNA.

17. The kit according to claim 15, further comprising a solid support that is contacted with a biological sample for which HMGA2 level is to be detected.

18. A device for the rapid detection of HMGA2 in a sample of bodily fluid, comprising an application zone for receiving a sample of bodily fluid; a labeling zone containing a binding agent that binds to HMGA2 in the sample; and a detection zone where HMGA2-bound binding agent is retained to give a signal, wherein the signal given for a sample from a subject with a HMGA2 level lower than a threshold concentration is different from the signal given for a sample from a patient with a HMGA2 level greater than a threshold concentration.

19. The device of claim 18, wherein the bodily fluid is urine or blood.

20. The device of claim 18, wherein the device has a reference zone which gives a signal which has the same intensity as the signal given in the detection zone for a sample from a subject having a HMGA2 level equal to the threshold concentration.

21. The method of claim 1, wherein an HMGA2 level in the sample that is approximately 10 times the HMGA2 levels of a control population that do not have ovarian cancer distinguishes a subject who has ovarian cancer from those who do not.
